(19) United States
(12) Patent Application Publication
(10) Pub. No.: US 2021/0282752 A1
(43) Pub. Date: Sep. 16, 2021

(54) ULTRASOUND IMAGING SYSTEMS AND METHODS

(71) Applicant: ZED MEDICAL, INC., Livermore, CA (US)

(72) Inventor: Joseph A. Jamello, Saratoga, CA (US)

(21) Appl. No.: 17/201,044

(22) Filed: Mar. 15, 2021

Related U.S. Application Data
(60) Provisional application No. 62/989,268, filed on Mar. 13, 2020.

(51) Int. Cl. A61B 8/00; A61B 8/12; A61B 8/08; G01S 15/89

(52) U.S. Cl. CPC A61B 8/54; A61B 8/4488; A61B 8/12; G01S 15/8997; A61B 8/5276; G01S 15/8915; A61B 8/5207

(57) ABSTRACT

An ultrasound imaging system includes an ultrasound transducer array having a plurality of transducer element and a catheter having one or more transmission lines programmably connected to the plurality of transducer elements. The programmable connection between the transmission lines and the plurality of transducer elements defines a synthetic aperture size. The ultrasound imaging system acquires images using an initial synthetic aperture size, detects a relative motion of a target of interest in the acquired images, and adjusts the synthetic aperture size based on the detected relative motion.

### CROSS-REFERENCE TO RELATED APPLICATIONS

This application claims priority to U.S. Provisional Patent Application No. 62/989,268, filed Mar. 13, 2020, the disclosure of which is hereby incorporated by reference in its entirety.

### BACKGROUND

Medical ultrasound imaging applications often involve imaging of targets of interest that are in motion relative to the imaging device such as cardiac motion, respiratory motion, and the like. Additionally, the imaging device may move relative to the targets of interest such as when a transducer is moved relative to an anatomical structure. Such relative motion can cause image artifacts such as misregistration and blurring. A clinician, such as a physician or sonographer, may have difficulty interpreting an image that contains image artifacts.

A general approach to reduce or eliminate motion artifacts is to minimize the scanning duration. This is often achieved by using high-channel count imaging systems that utilize ultrasound transducer arrays having high-channel count transmission lines. However, for some minimally invasive ultrasound imaging applications, such as intravascular ultrasound and endoscopy, device size constraints limit the number of transmission lines that can be housed in a catheter or endoscope. In such medical ultrasound imaging devices, the ultrasound transducer array element count can exceed the transmission line count of the catheter or endoscope.

Indirect scanning techniques may be used in which a single transmission line is connected to multiple ultrasound transducer array elements. The single transmission line can be used to sequentially transmit and receive on multiple ultrasound transducer array elements. However, this type of imaging sequence increases the scanning duration such that the indirect scanning techniques are sensitive to motion artifacts.

### SUMMARY

In general terms, the present disclosure relates to an ultrasound imaging system. In one possible configuration and by non-limiting example, the ultrasound imaging system adjusts a synthetic aperture size based on a detected relative motion.

In one aspect, an ultrasound imaging system comprises an ultrasound transducer array having a plurality of transducer elements, a catheter having one or more transmission lines programmably connected to the plurality of transducer elements, the programmable connection between the transmission lines and the plurality of transducer elements defining a synthetic aperture size, and a controller having at least one processing unit and a system memory storing instructions that, when executed by the at least one processor, causes the ultrasound imaging system to acquire images using an initial synthetic aperture size; detect a relative motion of a target of interest in the acquired images; and adjust the synthetic aperture size based on the detected relative motion.

The synthetic aperture size increases when the detected motion is less than a threshold value. In some examples, the synthetic aperture size increases by a factor of two when the detected motion is less than a threshold value. In some examples, the synthetic aperture size increases from 16-elements to 32-elements or from 32-elements to 64-elements based on the detected motion.

In some examples, the synthetic aperture size is not adjusted when the detected motion is greater than a threshold value. In some examples, the synthetic aperture size decreases when the detected motion is greater than a threshold value. In some examples, the synthetic aperture size decreases from 64-elements to 32-elements or from 32-elements to 16-elements based on the detected motion.

The relative motion of the target of interest is detected by generating an image pyramid for each acquired image, calculating pixel-wise and image-wise standard deviations from lower-level images of the image pyramids, and calculating motion weight factors from the image-wise standard deviations. The acquired images are filtered using motion weight factors. In some examples, a sequence of three images is acquired, an image pyramid is generated for each acquired image, and each image pyramid has three levels of images in which smoothing and sub sampling by a factor of two is repeated two times.

In another aspect, a method of acquiring ultrasound images comprises acquiring a sequence of images using an initial synthetic aperture size defined by a programmable connection between one or more transmission lines and a plurality of transducer elements; detecting a relative motion of a target of interest in the acquired images; maintaining the initial synthetic aperture size when the detected motion is greater than a threshold value; and increasing the initial synthetic aperture size when the detected motion is less than a threshold value. In some examples, the synthetic aperture size increases by a factor of two. The synthetic aperture size can increase from 16-elements to 32-elements or from 32-elements to 64-elements.

In some examples, the relative motion is detected by generating an image pyramid for each acquired image; calculating pixel-wise and image-wise standard deviations from lower-level images in each image pyramid; and calculating motion weight factors from the image-wise standard deviations. In some examples, the method further comprises filtering the acquired images using motion weight factors calculated from image-wise standard deviations of lower-level images in the image pyramids generated for each acquired image.

In another aspect, an ultrasound imaging system for optimizing ultrasound images of a moving target of interest comprises an ultrasound transducer array having a plurality of transducer elements; a catheter having one or more transmission lines operatively connected to the plurality of transducer elements in the ultrasound transducer array; and a controller having at least one processing unit and a system memory storing instructions that, when executed by the at least one processor, causes the ultrasound imaging system to acquire a sequence of images from the ultrasound transducer array; generate image pyramids for each acquired image; calculate pixel-wise and image-wise standard deviations from lower-level images of the image pyramids; calculate motion weight factors from the image-wise standard deviations; and filter the acquired images using motion weight factors.

In some examples, the ultrasound imaging system increases a synthetic aperture size defined between the one or more transmission lines and the plurality of transducer elements when there is an acceptable level of detected motion for the target of interest. In some examples, the synthetic aperture size increases by a factor of two. In some examples, the synthetic aperture size increases from 16-elements to 32-elements or from 32-elements to 64-elements.

In another aspect, a method of optimizing ultrasound images of a moving target of interest comprises acquiring a sequence of images; generating image pyramids for each acquired image; calculating standard deviations from lower-level images of the image pyramids; calculating motion weight factors from the standard deviations; and filtering the images using the calculated motion weight factors. In some examples, a sequence of three images is acquired, an image pyramid is generated for each acquired image, and each image pyramid has three levels smoothing and subsampling for each acquired image. In some examples, the image pyramids are constructed using a Gaussian average for smoothing and subsampling. In some examples, the image pyramids are Laplacian image pyramids in which a band-pass filter is applied to the acquired images. In some examples, the standard deviations include image-wise standard deviations calculated from pixel-wise standard deviations.

In another aspect, a method for creating a displacement map from ultrasound images of a target in motion comprises acquiring a sequence of images; creating sub-aperture images from each acquired image; generating image pyramids for each sub-aperture image; calculating tissue displacement from lower-level images in each image pyramid; and creating a displacement map using the calculated tissue displacements.

In another aspect, a method for interpolating an image of a target in motion comprises acquiring a sequence of images; creating sub-aperture images from each acquired image; generating image pyramids for each sub-aperture image; calculating tissue displacements from lower-level images in each image pyramid; generating interpolated sub-aperture images using the calculated tissue displacements; and creating an interpolated full image from the interpolated sub-aperture images.

### BRIEF DESCRIPTION OF THE DRAWINGS

The following drawing figures, which form a part of this application, are illustrative of the described technology and are not meant to limit the scope of the disclosure in any manner.

FIG. 1 illustrates an example of a first ultrasound image with a target of interest and a surrounding tissue.

FIG. 2 illustrates examples of a first Level 0 ultrasound image, a first Level 1 ultrasound image, and a first Level 2 ultrasound image.

FIG. 3 illustrates an example first image pyramid of the first Level 0 ultrasound image, the first Level 1 ultrasound image, and the first Level 2 ultrasound image.

FIG. 4 illustrates a sequence of an example first ultrasound image, an example second ultrasound image, and an example third ultrasound image.

FIG. 5 illustrates an example sequence of image pyramids including an example first image pyramid for a first ultrasound image, an example second image pyramid for a second ultrasound image, and an example third image pyramid for a third ultrasound image.

FIG. 6 illustrates an example of a standard deviation image.

FIG. 7 illustrates an example method for filtering an ultrasound image using image pyramids in accordance with certain example embodiments of the present application.

FIG. 8 illustrates an example first ultrasound image that includes a target of interest at a first position and a surrounding tissue.

FIG. 9 illustrates an example second ultrasound image that includes a target of interest at a second position and a surrounding tissue.

FIG. 10 illustrates an example third ultrasound image that includes a target of interest at a third position and a surrounding tissue.

FIG. 11 illustrates an example of an ultrasound transducer array used to image a target.

FIG. 12 illustrates an example method for filtering an image based on a detected level of motion in accordance with certain example embodiments of the present application.

FIG. 13 illustrates an ultrasound transducer array used to acquire an example of a first ultrasound image.

FIG. 14 illustrates an ultrasound transducer array used to acquire an example of a second ultrasound image.

FIG. 15 illustrates an ultrasound transducer array used to acquire an example of a third ultrasound image.

FIG. 16 illustrates an example of a first ultrasound image, second ultrasound image, and third ultrasound image each segmented into sub-aperture images.

FIG. 17 illustrates an example time-lapse image that shows a change in position of a target of interest at a first position, a second position, and a third position.

FIG. 18 illustrates an example displacement map that includes a position grid and optical flow where magnitude and direction of motion is represented by length and direction of arrows.

FIG. 19 illustrates an example method for creating a displacement map from an image sequence in accordance with certain example embodiments of the present application.

FIG. 20 illustrates example sub-aperture images of an ultrasound image.

FIG. 21 illustrates an example method for calculating an interpolated ultrasound image from sequentially acquired ultrasound images in accordance with certain example embodiments of the present application.

FIG. 22 is a block diagram schematically illustrating an ultrasound imaging system.

FIG. 23 is a block diagram illustrating physical components of a controller.

### DETAILED DESCRIPTION

This patent application is directed to medical imaging devices and methods that detect motion in order to minimize motion-based image artifacts and to improve image quality.

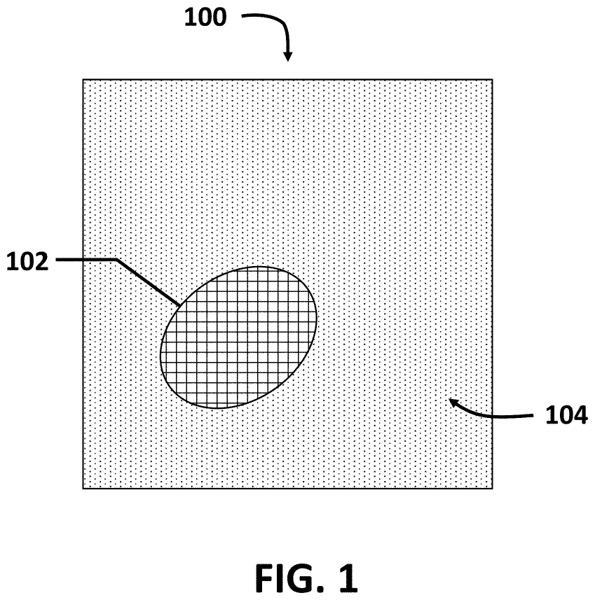

FIG. 1 illustrates a first ultrasound image 100 having a target of interest 102 and a surrounding tissue 104. In the example illustrated in FIG. 1, the first ultrasound image 100 has a size (also referred to as resolution) that is square such that the image width is the same as the image height. In some examples, the size of the first ultrasound image 100 is between 50 pixels and 5000 pixels. In some further examples, the first ultrasound image 100 can have a size corresponding to a gradation of 100 pixels such as 100 pixels, 200 pixels, 300 pixels, 400 pixels, 500 pixels, and the like. Image size may depend on multiple factors including the type of imaging device and the type of scan geometry used, as well as the imaging target. In some examples, the size of the first ultrasound image 100 is non-square such that the image width of the first ultrasound image 100 is not the same as the image height.

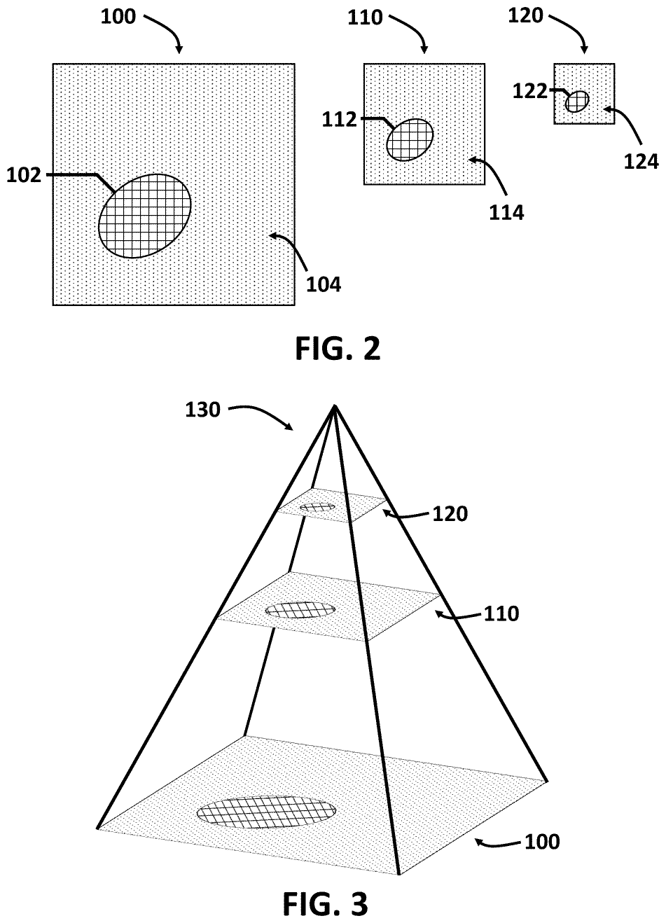

FIG. 2 shows the first ultrasound image 100 (also referred to as the first Level 0 ultrasound image), a first Level 1 ultrasound image 110 that is a smoothed and subsampled version of the first ultrasound image 100, and a first Level 2 ultrasound image 120 that is a smoothed and subsampled version of the first Level 1 ultrasound image 110. The first Level 1 ultrasound image 110 includes a target of interest 112 and a surrounding tissue 114. The first Level 2 ultrasound image 120 includes a target of interest 122 and a surrounding tissue 124.

FIG. 3 illustrates a first image pyramid 130 of the first Level 0 ultrasound image 100, the first Level 1 ultrasound image 110, and the first Level 2 ultrasound image 120. In this example, the first image pyramid 130 has three levels in which the cycle of smoothing and subsampling by a factor of two is repeated two times. In some examples, a Gaussian (or lowpass) pyramid is constructed by using a Gaussian average for smoothing and subsampling by a factor of two. As an illustrative example, the first Level 0 ultrasound image 100 can have an image size of 256 pixels by 256 pixels, the first Level 1 ultrasound image 110 can have an image size of 128 pixels by 128 pixels, and the first Level 2 ultrasound image 120 can have an image size of 64 pixels by 64 pixels. Advantageously, the smoothing and subsampling performed by the first image pyramid 130 on the first Level 0 ultrasound image 100 requires less computation resources and computation time by reducing the image processing on the smaller-sized first Level 2 ultrasound image 120. It is contemplated that in other examples, the image pyramid can have a different number of levels in which the cycle of smoothing and subsampling is performed.

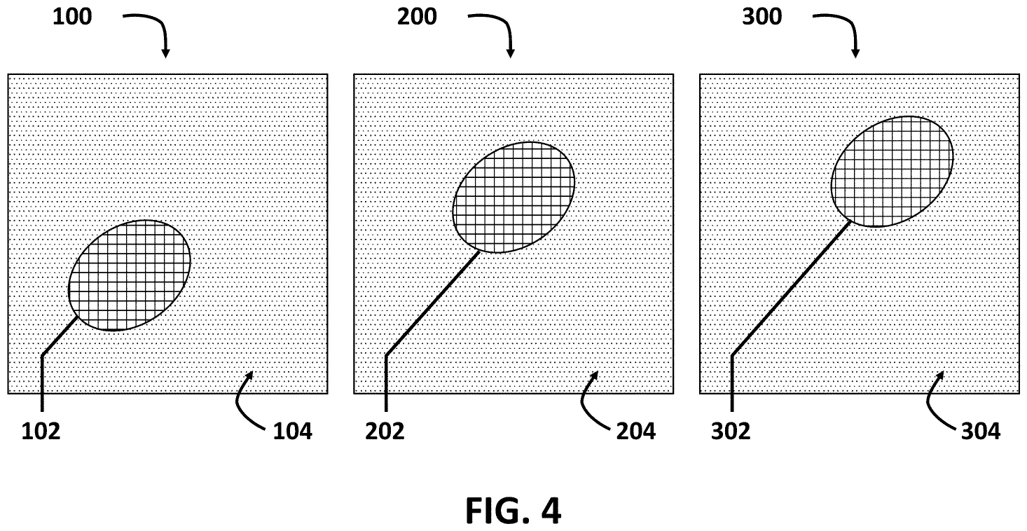

For motion detection, image pyramids such as the first image pyramid 130 of FIG. 3 are generated for consecutive images. FIG. 4 includes the first ultrasound image 100 with the target of interest 102 and surrounding tissue 104, a second ultrasound image 200 with a target of interest 202 and a surrounding tissue 204, and a third ultrasound image 300 with a target of interest 302 and a surrounding tissue 304. The first ultrasound image 100 is acquired prior to the second ultrasound image 200. The second ultrasound image 200 is acquired prior to the third ultrasound image 300. As illustrated in FIG. 4, the targets of interest 102, 202, 302 represent the same target at different locations which indicates relative motion of the target between the first ultrasound image 100, the second ultrasound image 200, and the third ultrasound image 300.

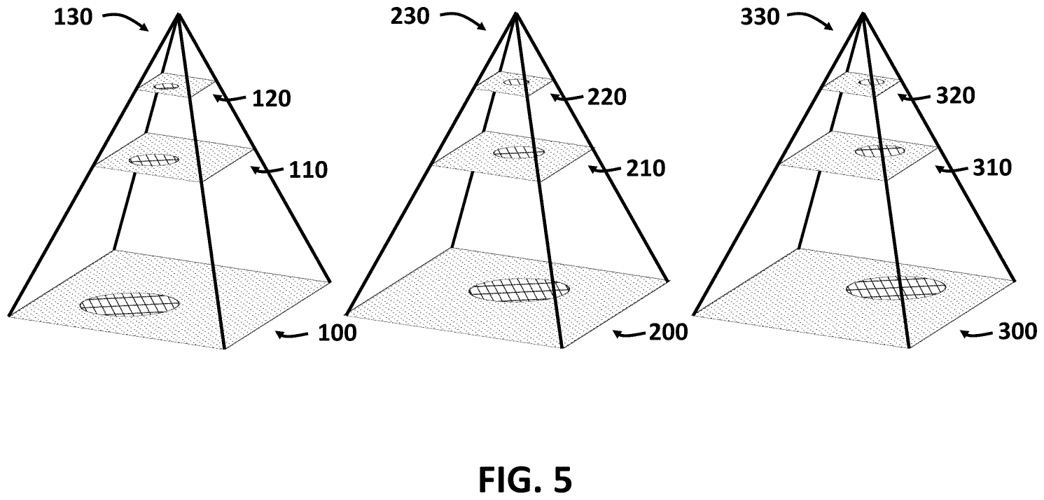

The use of image pyramids enables the detection of motion of a target. FIG. 5 shows the first image pyramid 130 for the first ultrasound image 100, a second image pyramid 230 for the second ultrasound image 200 (also referred to as a second Level 0 ultrasound image), and a third image pyramid 330 for the third ultrasound image 300 (also referred to as a third Level 0 ultrasound image). The first image pyramid 130 includes the first Level 0 ultrasound image 100, the first Level 1 first ultrasound image 110, and the first Level 2 ultrasound image 120 that are shown in FIG. 3. The second image pyramid 230 includes the second Level 0 ultrasound image 200, a second Level 1 ultrasound image 210, and a second Level 2 ultrasound image 220. The third image pyramid 330 includes the third Level 0 ultrasound image 300, a third Level 1 ultrasound image 310, and a third Level 2 ultrasound image 320.

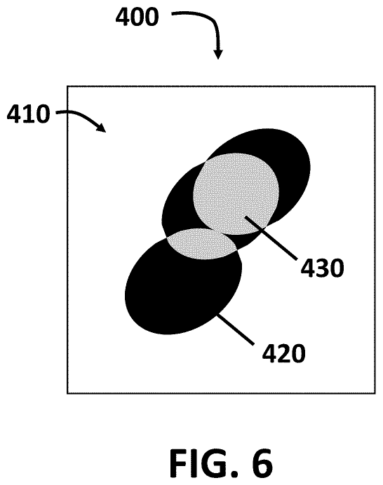

Motion of the target of interest is detected using the lowest resolution images of the image pyramids 130, 230, 330, namely the Level 2 images 120, 220, 320. In some examples, the motion of the target of interest is detected by measuring standard deviations. For example, a pixel-wise standard deviation is calculated from the three Level 2 images 120, 220, 320 such that a standard deviation is calculated from the image values at each pixel location to generate a standard deviation image 400 as shown in FIG. 6. Pixel locations 410 where pixel values are substantially similar have relatively small standard deviation values. Pixel locations 420 where pixel values are substantially different have relatively large standard deviation values. Pixel locations 430 where pixel values are only modestly different have relatively modest standard deviation values. The range of standard deviation values that are considered small, modest, and large can be empirically determined based on the particular imaging application.

In some illustrative examples, the Level 2 images 120, 220, 320 have 8-bit pixel values that range approximately between 0 and 255 pixel values. In medical ultrasound imaging, larger pixel values generally correspond to anatomical regions that include stronger acoustic scatterers and reflectors, such as tissue boundaries, fibrous tissue, and calcified tissue. Smaller pixel values generally correspond to anatomical regions that include weaker acoustic scatterers and reflectors, such as fluid-filled cysts and lipid-rich plaques. The pixel values of the surrounding tissue are approximately 64 and, as an illustrative example, may correspond to connective tissue. The pixel values of the target of interest are approximately 128 and, as an illustrative example, may correspond to a heterogeneous bronchial lymph node.

Pixels in the Level 2 images 120, 220, 320 that are in the surrounding tissue region in all images have pixel-wise standard deviation values that are substantially close to 0. Similarly, pixels in the Level 2 images 120, 220, 320 that are in the target of interest region in all images have pixel-wise standard deviation values that are substantially close to 0. Pixels of the Level 2 images 120, 220, 320 that change from the surrounding tissue region to the target of interest region or from the target of interest region to the surrounding tissue region have pixel-wise standard deviation values in the range of approximately 35 and 40.

An image-wise standard deviation (σ) can be calculated as the root-mean-square (RMS) of the pixel-wise standard deviation values. In some examples, the calculated image-wise standard deviation can be compared to a motion detection threshold value to classify the motion of the target of interest. As an illustrative example, a pixel-wise standard deviation value between 5 and 20 (e.g., 15) can be selected as a motion detection threshold having a high degree of sensitivity. As another illustrative example, a pixel-wise standard deviation value between 20 and 35 (e.g., 30) can be selected as a motion detection threshold having less sensitivity.

In some examples, information from neighboring ultrasound images and motion weight factors can be used to filter an ultrasound image based on the degree of motion. In general, image filtering can be more aggressive in cases of less motion where the same anatomy is present in a sequence of images (e.g., tissue type, location, and appearance are substantially the same). Image filtering can be less aggressive in cases of more motion where the anatomy varies in a sequence of images (e.g., tissue type, location, or appearance is not substantially the same).

The motion weight factors are calculated using the image-wise standard deviation value and are applied to each ultrasound image. In some examples, the motion weight factors are normalized to avoid scaling the pixel values of a filtered image. The motion weight factor values can depend on the particular clinical application and can be empirically determined. As an illustrative example, a first motion weight factor value (f₁) that is applied to the first Level 0 ultrasound image 100 is defined as 0.33 for 0 a 1, 0.33× (25−σ)/24 for 1<σ≤25, and 0 for σ>25, and a standard deviation threshold of 25 represents a high level of motion above which no frame filtering is used. As another example, a third motion weight factor value (f₃) that is applied to the third Level 0 ultrasound image 300 is equal to f₁.

As another illustrative example, a second motion weight factor (f₂) that is applied to the second Level 0 ultrasound image 200 is equal to 1−(f₁+f₃). The motion weight factor values for the neighboring images (f₁, f₃) are larger for smaller standard deviation values which correspond to less motion. The sum of the three motion weight factors is 1. As an example of aggressive filtering in a case of low motion (σ≤1), the motion weight factors f₁=f₃=0.33 and f₂=0.34. A filtered second Level 0 ultrasound image is calculated from the first Level 0 ultrasound image (I₁) 100, second Level 0 ultrasound image (I₂) 200, third Level 0 ultrasound image (I₃) 300, and the motion weight factors (f₁, f₂, f₃) as f₁×I₁+f₂×I₂+f₃×I₃. The contribution of the first Level 0 ultrasound image and third Level 0 ultrasound image to the filtered image is substantially the same as the second Level 0 ultrasound image. As an example of no filtering in a case of high motion σ>25), the motion weight factors f₁=f₃=0 and f₂=1. A filtered second Level 0 ultrasound image is equivalent to the second Level 0 ultrasound image (I₂) 200. The first Level 0 ultrasound image and third Level 0 ultrasound image do not contribute to the filtered image.

In some examples, the second Level 0 ultrasound image 200 is filtered using the first Level 0 ultrasound image 100, the third Level 0 ultrasound image 300, and the motion weight factor. Each pixel value of the filtered second Level 0 ultrasound image is calculated as a sum of the corresponding pixel value multiplied by the motion weight factor value of each image, or written in mathematical notation as:

$${\sum }_{n = 1}^{3} {f}_{n} {p}_{ij}$$

wherein fₙ is motion factor of the n^{th} image and pᵢⱼ is the value of the pixel at the ij^{th} location (or i^{th} column and j^{th} row).

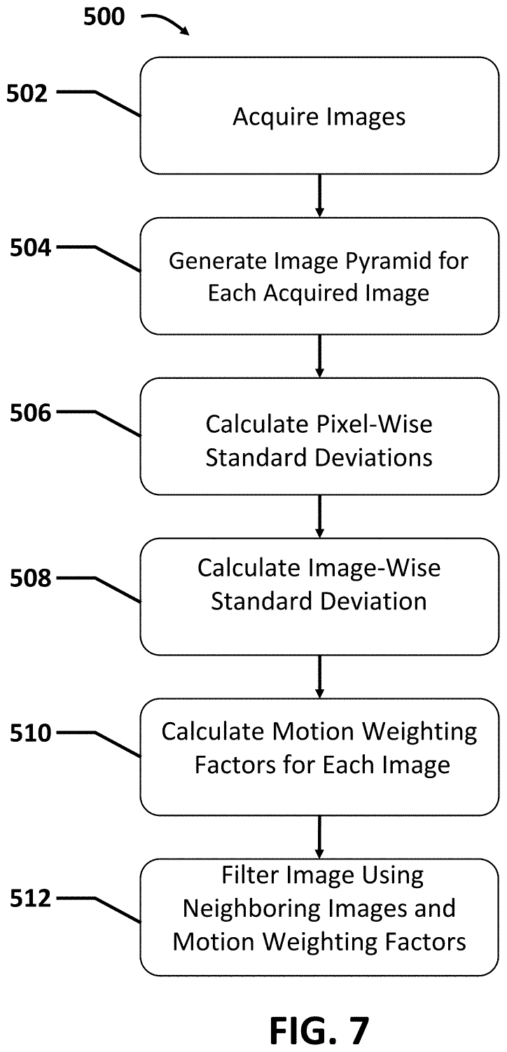

FIG. 7 illustrates a method 500 for filtering an ultrasound image using image pyramids. The method 500 includes an operation 502 of acquiring a plurality of ultrasound images. In some examples, three ultrasound images are acquired. In other examples, more than three images or fewer than three images are acquired at operation 502.

Next, an operation 504 includes generating image pyramids for each of the acquired images. In examples where three ultrasound images are acquired in operation 502, three image pyramids (one for each acquired ultrasound image) are generated at operation 504. In some examples, each image pyramid includes three levels of smoothing and subsampling using a Level 0 ultrasound image, a Level 1 ultrasound image, and a Level 2 ultrasound image. In these examples, the three levels of smoothing and subsampling is done by a factor of two and is repeated two times. In other examples, more than or fewer than three levels of smoothing and subsampling is done. In some examples, a Gaussian image pyramid is constructed by using a Gaussian average for smoothing and subsampling by a factor of two. In these examples, a low-pass filter is applied using the Gaussian image pyramid. In other examples, different image pyramids can be used such as a Laplacian image pyramid in which a band-pass filter is applied.

The method 500 includes an operation 506 of calculating pixel-wise standard deviations from the Level 2 images. Next, the method 500 includes an operation 508 of calculating an image-wise standard deviation from the pixel-wise standard deviations. Thereafter, the method 500 includes an operation 510 of calculating motion weight factors for each acquired image using the image-wise standard deviation. The motion weight factors can be calculated in accordance with the examples described above. Next, an operation 512 is performed to filter a second Level 0 image using the first and third Level 0 images and the motion weight factors. As an illustrative example, each pixel value of the filtered second Level 0 ultrasound image is calculated as a sum of the corresponding pixel value multiplied by the motion weighting factor value of each image.

In view of the foregoing description of the method 500, the acquired images may include a different number of distinct regions, pixel values for the regions, relative levels of motion for the regions, and ranges of pixel-wise standard deviation values. These different parameters will affect the resultant motion weight factors and the degree of filtering of an acquired image.

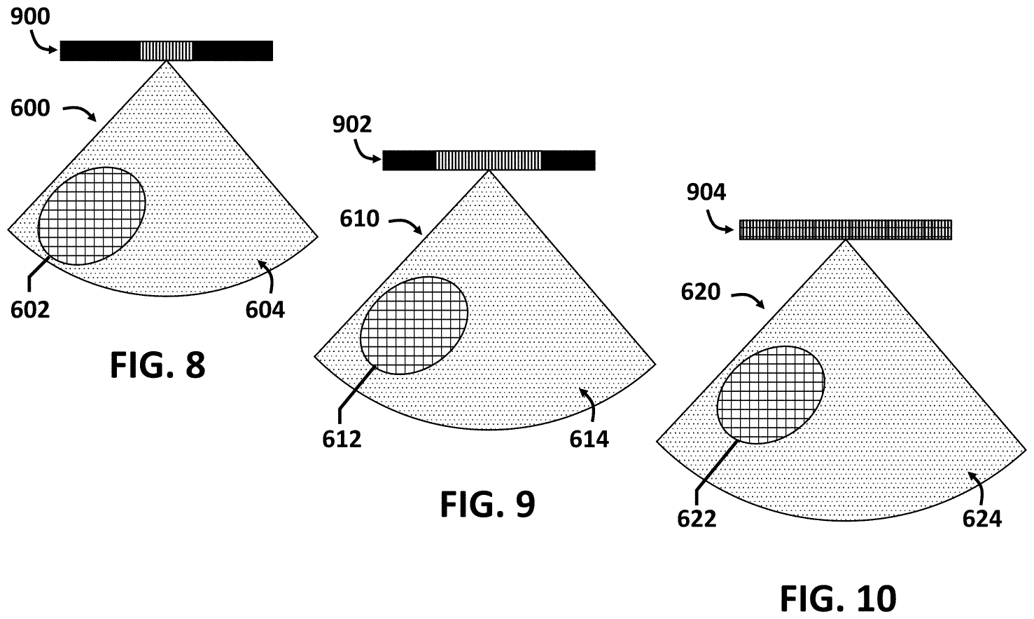

In another example embodiment in accordance with the present application, motion of the target of interest is detected using an imaging sequence in which an aperture of an ultrasound transducer array expands. Referring now to FIGS. 8, 9, and 10, a first ultrasound image 600 that includes a target of interest at a first position 602 and a surrounding tissue 604 is constructed using an ultrasound transducer array 900 having 16 active transducer elements. A second ultrasound image 610 that includes a target of interest at a second position 612 and a surrounding tissue 614 is constructed using an ultrasound transducer array 902 having 32 active transducer elements and an expanded aperture. A third ultrasound image 620 that includes a target of interest at a third position 622 and a surrounding tissue 624 is constructed using an ultrasound transducer array 904 having 64 active transducer elements and a further expanded aperture.

The targets of interest at the first, second, and third positions 602, 612, 622 represent substantially the same anatomy. The surrounding tissues 604, 614, 624 represent substantially the same anatomy. The depth of penetration of an ultrasound image generally increases with increasing aperture size of the ultrasound transducer array. Thus, the third ultrasound image 620 that is constructed using the ultrasound transducer array 904 having 64 transducer elements has a larger depth of penetration than the first and second ultrasound images 600, 610.

Generally, a synthetic aperture size is defined by the transducer elements, one or more transmission lines, and a programmable connection between the one or more transmission lines and transducer elements during a transmit sequence and/or receive sequence. For example, a transmission line can be programmably connected to multiple ultrasound transducer array elements such that the transmission line is used to sequentially transmit and receive on the multiple ultrasound transducer array elements. In one example embodiment of the present application, a synthetic aperture ultrasound imaging system is programmed to perform a cascading imaging sequence to optimize the number of transmit and receive events based on detected motion of a target of interest in order to optimize image quality while reducing image artifacts that result from the motion of the target of interest during an ultrasound scan.

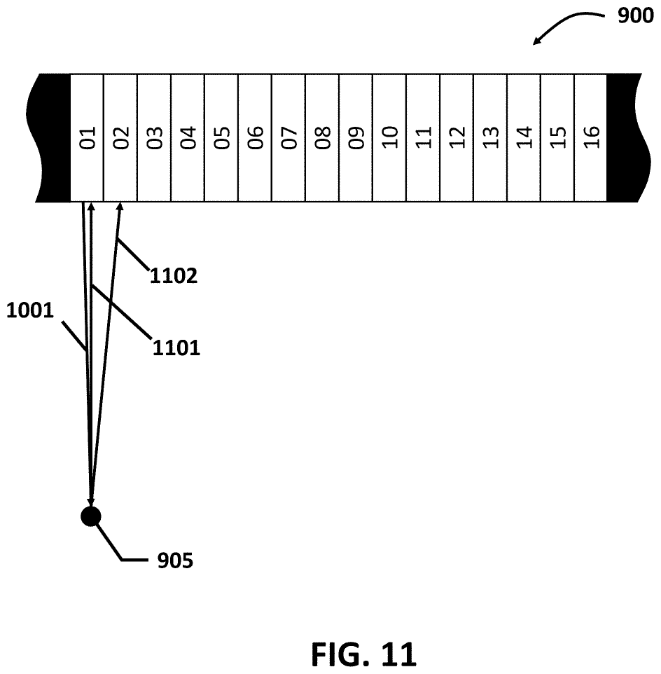

FIG. 11 is an illustrative example of a synthetic aperture ultrasound imaging system having an ultrasound transducer array 900 with 16 transducer elements that are used to image a target 905. The 16 individual ultrasound transducer elements are labeled from 1 to 16. The complete data set for a synthetic aperture imaging system includes transmit and receive events for each pair of ultrasound transducer elements acting as a transmitter (Tx) and receiver (Rx).

The transmit-receive event Tx01Rx01 represents a transmit event 1001 from a first ultrasound transducer element 1 to the target 905 and a receive event 1101 from the target 905 to the first ultrasound transducer element 1. Similarly, the transmit-receive event Tx01Rx02 represents a transmit event 1001 from the first ultrasound transducer element 1 to the target 905 and a receive event 1102 from the target 905 to a second ultrasound transducer element 2.

The complete data set for the synthetic aperture ultrasound imaging system including the 16-element ultrasound transducer array 900 requires 256 transmit-receive events to produce a single image or frame. In some examples when acoustic reciprocity is available, the complete data set for the synthetic aperture ultrasound imaging system having the 16-element ultrasound transducer array 900 requires 136 transmit-receive events. For example, acoustic reciprocity means that the transmit-receive event Tx01Rx02 is equivalent to Tx02Rx01.

As the aperture of the transducer increases from 16 elements to 32 elements to 64 elements, the image quality improves due to increased penetration, however, the number of transmit-receive events required to complete a single image or frame increases almost quadratically from 136 transmit-receive events to 528 transmit-receive events to 2080 transmit-receive events when acoustic reciprocity is available. It is advantageous to minimize the number of transmit-receive events to reduce the scan duration when there is high level of motion in order to reduce image artifacts that may result from the high level of motion. Additionally, it is advantageous to maximize the number of transmit-receive events when there is a low level of motion in order to enhance image quality by providing deeper penetration.

In some examples, the synthetic aperture ultrasound imaging system is adapted to use more than one receive channel to reduce the scan duration (e.g., time). For example, synthetic aperture imaging on a 64-element ultrasound imaging system using one receive channel can generate about 7 to 8 frames per second, whereas synthetic aperture imaging on a 64-element ultrasound imaging system using four receive channels can generate about 30 frames per second for “real-time” imaging. Thus, in some examples, the synthetic aperture ultrasound imaging system transmits on one element and receives on four elements until all of the unique non-reciprocal combinations of transmit and receive events are completed to generate a frame.

In imaging applications where the level of motion of a target of interest is not known a priori or the level of motion changes during an imaging session, the imaging system can cascade from a synthetic aperture with a smaller number of programmably connected transducer elements to a synthetic aperture with a higher number of programmably connected transducer elements.

In one example embodiment, a synthetic aperture imaging sequence includes image acquisition first by a 16-element synthetic aperture, followed by image acquisition by a 32-element synthetic aperture when motion is low during the image acquisition by the 16-element synthetic aperture, and followed by image acquisition by a 64-element synthetic aperture when motion is low during the image acquisition by the 32-element synthetic aperture. When high levels of motion are detected during image acquisition, a smaller synthetic aperture is used (e.g., the 16-element or 32-element synthetic apertures) that enables higher imaging frame rates to reduce motion impacts on image quality.

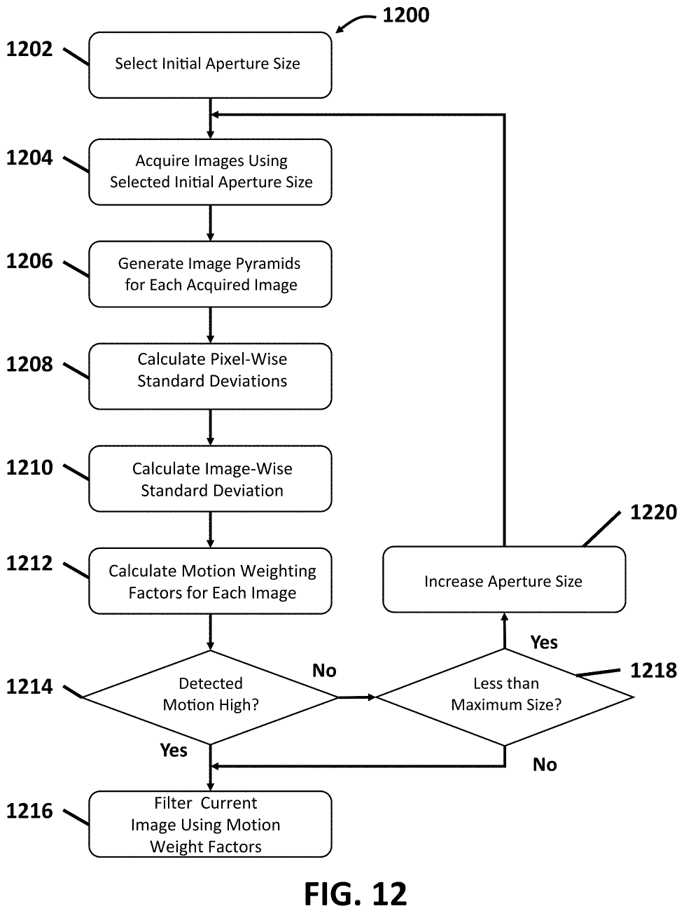

In one example embodiment of the present application, an ultrasound transducer array having 64 transducer elements is used to perform synthetic aperture imaging by performing a cascading imaging sequence. FIG. 12 illustrates a method 1200 for performing a cascading imaging sequence that cascades from a 16-element synthetic aperture to a 64-element synthetic aperture based on a detected level of motion during an ultrasound scan.

The method 1200 includes an operation 1202 of selecting an initial synthetic aperture size for the ultrasound transducer array. In some examples, the initial synthetic aperture size is 16 transducer elements. It is contemplated that the initial synthetic aperture size may vary such that it may be fewer than 16 transducer elements or more than 16 transducer elements.

Next, the method 1200 includes an operation 1204 of acquiring a plurality of ultrasound images using the initial synthetic aperture size. In some examples, three ultrasound images using the initial synthetic aperture size are acquired during operation 1204. In other examples, more than three ultrasound image or fewer than three ultrasound images are acquired.

Next, operation 1206 includes generating image pyramids for each of the acquired ultrasound images. In examples where three ultrasound images are acquired in operation 1204, three image pyramids (one for each acquired ultrasound image) are generated at operation 1206. In some examples, each image pyramid includes three levels of smoothing and subsampling using a Level 0 ultrasound image, a Level 1 ultrasound image, and a Level 2 ultrasound image. In these examples, the three levels of smoothing and subsampling is done by a factor of two and is repeated two times. In other examples, more than or fewer than three levels of smoothing and subsampling is done. In some examples, a Gaussian image pyramid is constructed by using a Gaussian average for smoothing and subsampling by a factor of two. In these examples, a low-pass filter is applied using the Gaussian image pyramid. In other examples, different image pyramids can be used such as a Laplacian image pyramid in which a band-pass filter is applied.

Next, the method 1200 includes an operation 1208 of calculating pixel-wise standard deviations from the Level 2 ultrasound images of the image pyramids. Next, the method 1200 includes an operation 1210 of calculating a Level 2 image-wise standard deviation from the pixel-wise standard deviations calculated from operation 1208. A further operation 1212 is performed to calculate motion weight factors for each Level 2 image.

Next, the method 1200 includes an operation 1214 of detecting a motion of the target of interest and comparing the detected motion to a threshold value. In some examples, the motion is detected in accordance with the one or more examples described above.

When the detected motion is greater than the threshold value such that the detected motion is high (i.e., “Yes” at operation 1214), the method 1200 proceeds to operation 1216 of filtering the acquired images using the motion weight factors. In some examples, when the detected motion is greater than the threshold value, the synthetic aperture size is not adjusted.

Alternatively, when the detected motion is less than the threshold value such that the detected motion is low, the method 1200 proceeds to an operation 1218 that includes determining whether the current synthetic aperture size is less than a maximum synthetic aperture size. In accordance with the example described above, the maximum synthetic aperture size is 64 transducer elements. In other examples, the maximum synthetic aperture size may be fewer than 64 transducer elements or more than 64 transducer elements.

When the current synthetic aperture size is less than the maximum synthetic aperture size (i.e., “Yes” at operation 1218), the method 1200 proceeds to an operation 1220 such that the synthetic aperture size is increased. In some examples, the synthetic aperture size is increased by a factor of two. As an illustrative example, when the initial synthetic aperture size is 16-elements, operation 1218 determines that the current 16-element synthetic aperture size is less than the maximum synthetic aperture size of 64-elements (i.e., “Yes” at operation 1218) such that operation 1220 increases the current synthetic aperture size from 16-elements to 32-elements. As another illustrative example, when the initial synthetic aperture size is 32-elements, operation 1218 determines that the current 32-element synthetic aperture size is less than the maximum synthetic aperture size of 64-elements (i.e., “Yes” at operation 1218) such that operation 1220 increases the current synthetic aperture size from 32-elements to 64-elements. As a further example, when the initial synthetic aperture size is 64-elements, operation 1218 determines that the current 64-element synthetic aperture size is equal to the maximum 64-element synthetic aperture size (i.e., “No” at operation 1218) such that the method 1200 does not adjust the synthetic aperture size. Instead, the method 1200 proceeds to operation 1216 of filtering the ultrasound images using the calculated motion weight factors.

In some examples, the method 1200 repeats operations 1204 to 1220 after completion of operation 1220. As an illustrative example, after the initial synthetic aperture size increases from 16-elements to 32-elements, the method 1200 repeats operations 1204 to 1220. When the detected motion is determined to be high at operation 1214 in the ultrasound images acquired with the 32-element synthetic aperture (i.e., “Yes” at operation 1214), the method proceeds to filter the ultrasound images at operation 1216. When the motion level is determined to be low at operation 1214 (i.e., “No” at operation 1214), the method 1200 proceeds to operation 1218 to compare the current synthetic aperture size of 32-elements to the maximum synthetic aperture size of 64-elements. Since the current synthetic aperture size of 32 transducer elements is less than maximum size of 64 transducer elements (i.e., “Yes” at operation 1218), operation 1220 is repeated such that the synthetic aperture size is increased from 32-elements to 64-elements.

After repeating operation 1220, the operations 1204 to 1214 are repeated for a second time. As an illustrative example, after the synthetic aperture size increases from 32 transducer elements to 64 transducer elements, the method 1200 repeats operations 1204 to 1220. When the detected motion is determined to be high at operation 1214 in the ultrasound images acquired with the 64-element synthetic aperture (i.e., “Yes” at operation 1214), the method 1200 proceeds to filter the ultrasound images at operation 1216. When the motion level is determined to be low at operation 1214 (i.e., “No” at operation 1214), the method 1200 proceeds to operation 1218 to compare the current synthetic aperture size of 64-elements to the maximum synthetic aperture size of 64-elements. Since the synthetic aperture size of 64 transducer elements is equal to the maximum aperture size of 64 transducer elements (i.e., “No” at operation 1218), the method 1200 does not adjust the synthetic aperture size, and proceeds to operation 1216 to filter the ultrasound images acquired using the increased synthetic aperture size of 64 transducer elements.

It is contemplated that different synthetic aperture sizes may be selected at operation 1202, a different number of ultrasound images may be acquired at operation 1204 (e.g., more than or fewer than three ultrasound images), the image pyramids generated at operation 1206 may have a different number of levels and may be generated using different techniques (e.g., by using a Laplacian filter) to create various multi-level image pyramids, and different standard deviation thresholds may be used to calculate the motion weight factors.

In addition, it is contemplated that in certain example embodiments, the method 1200 may include an optional step of reducing the synthetic aperture size in response to determining that the detected motion is high at operation 1214. As an example, when the current synthetic aperture size is 64-elements and the detected motion is determined to be high at operation 1214, the method 1200 may include a further step of reducing the synthetic aperture size from 64-elements to 32-elements. Thereafter, the method 1200 may proceed to filter the ultrasound images that were acquired using the 64-element synthetic aperture size and repeat operations 1204 to 1214 using the reduced synthetic aperture size of 32-elements. As a further illustrative example, when the current synthetic aperture size is 32-elements and the detected motion is determined to be high at operation 1214, the method 1200 may include a further step of reducing the synthetic aperture size from 32-elements to 16-elements. Thereafter, the method 1200 may proceed to filter the ultrasound images acquired from the 32-element synthetic aperture size and repeat operations 1204 to 1214 using a reduced synthetic aperture size of 16-elements.

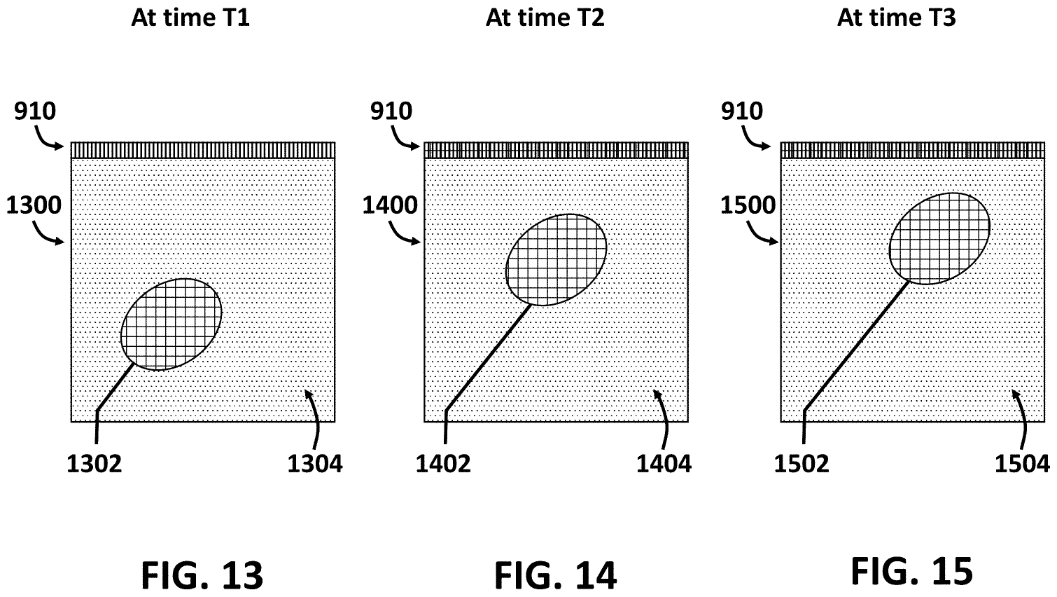

In still another example embodiment in accordance with the present application, motion estimation can be used to create displacement maps that provide a visualization of tissue motion. Referring now to FIGS. 13, 14, and 15, an ultrasound transducer array 910 is used to acquire a first ultrasound image 1300 at time T1. The first ultrasound image 1300 includes a target of interest 1302 at a first position and a first surrounding tissue 1304. The ultrasound transducer array 910 is used to acquire a second ultrasound image 1400 at time T2 that includes a target of interest 1402 at a second position and a second surrounding tissue 1404. The ultrasound transducer array 910 is further used to acquire a third ultrasound image 1500 at time T3 that includes a target of interest 1502 at a third position and a third surrounding tissue 1504. In this example, Time T1 occurs before time T2, and Time T2 occurs before time T3.

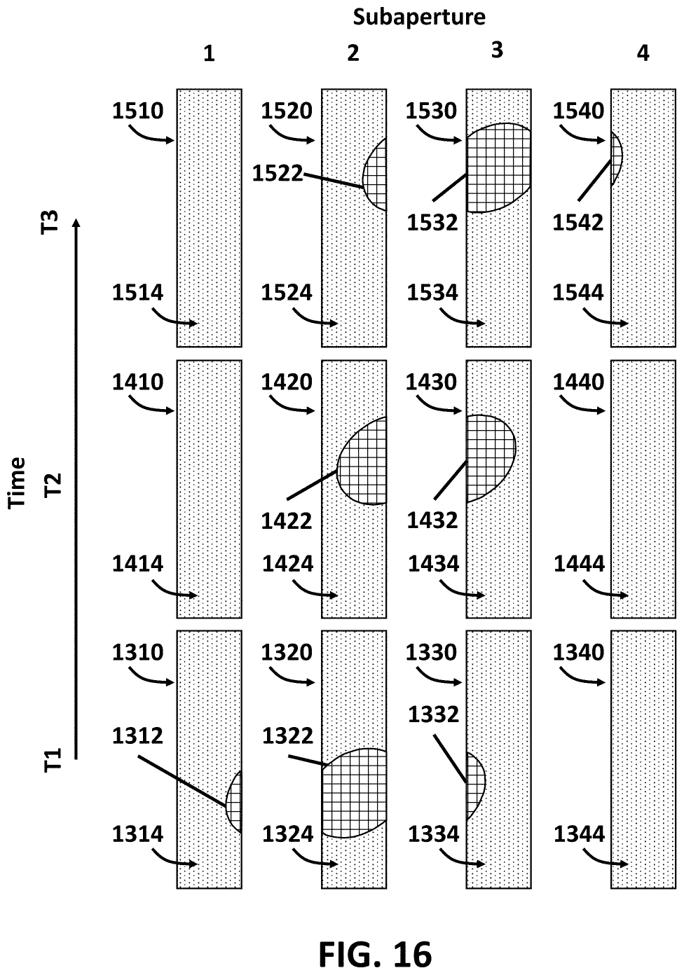

Referring now to FIG. 16, the first ultrasound image 1300, second ultrasound image 1400, and third ultrasound image 1500 are each segmented into sub-aperture images. In general, the sub-aperture image is a segmented portion of the whole image. In the example illustrated in FIG. 16, each ultrasound image is segmented into four sub-aperture images. For example, the first ultrasound image 1300 at time T1 is segmented into four sub-aperture images 1310, 1320, 1330, and 1340. The target of interest 1302 at the first position is segmented into target of interest segments 1312, 1322, and 1332. The first surrounding tissue 1304 is segmented into segmented first surrounding tissues 1314, 1324, 1334, and 1344.

Similarly, the second ultrasound image 1400 at time T2 is segmented into four sub-aperture images 1410, 1420, 1430, and 1440. The target of interest 1402 at the second position is segmented into target of interest segments 1422 and 1432. The second surrounding tissue 1404 is segmented into segmented second surrounding tissues 1414, 1424, 1434, and 1444.

The third ultrasound image 1500 at time T3 is segmented into four sub-aperture images 1510, 1520, 1530, and 1540. The target of interest 1502 at the third position is segmented into target of interest segments 1522, 1532, and 1542. The third surrounding tissue 1504 is segmented into segmented third surrounding tissues 1514, 1524, 1534, and 1544.

In other example embodiments, it is contemplated that each ultrasound image can be segmented into a different number of sub-aperture images such that each ultrasound image can be segmented into more than or fewer than four sub-aperture images.

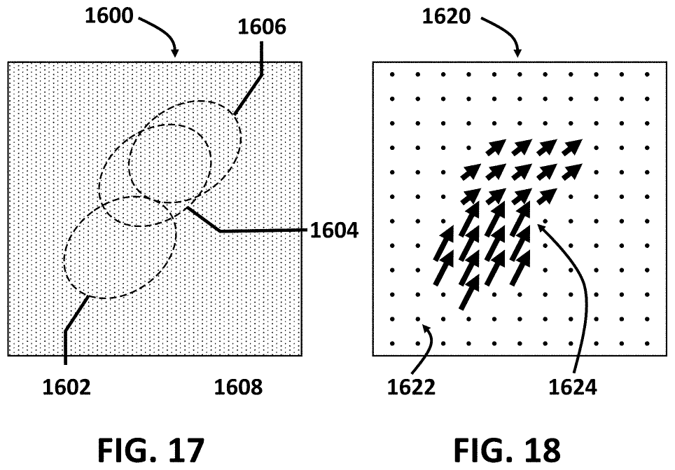

FIG. 17 illustrates an example time-lapse image 1600 that shows a change in position of a target of interest at a first position 1602, a second position 1604, and a third position 1606 as well as surrounding tissue 1608. FIG. 18 illustrates an example displacement map 1620 that includes a position grid 1622 and flow pattern 1624 in which the magnitude and direction of motion is represented by length and direction of arrows. Image pyramids that are generated from the sub-aperture images can be used to create the displacement map 1620. Also, the motion of the target of interest may be estimated using image processing techniques in which relative motion of pixel patterns are estimated from a sequence of images.

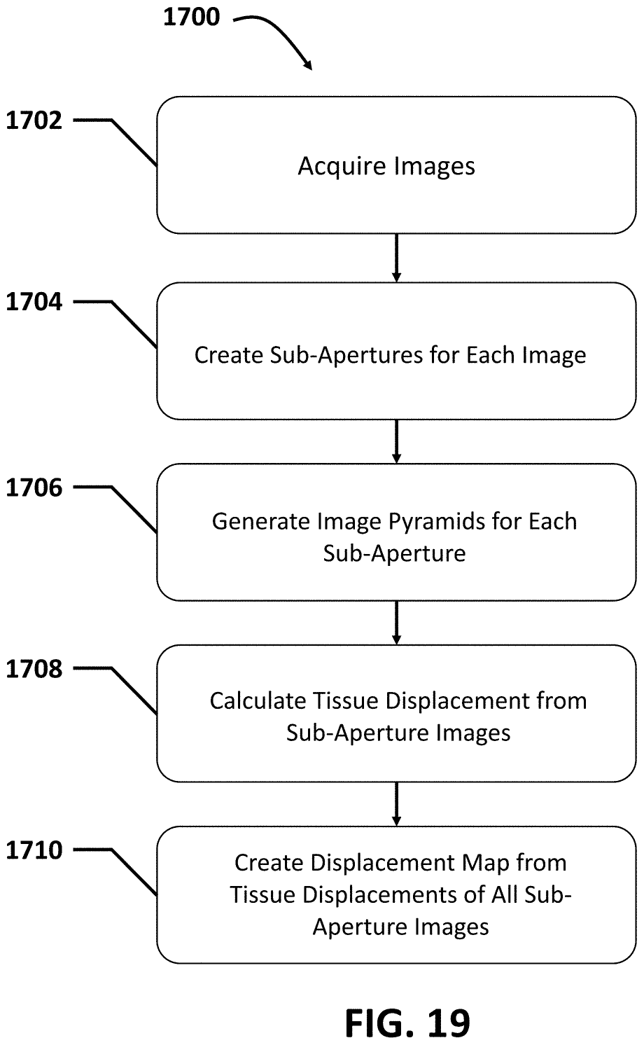

FIG. 19 illustrates a method 1700 for creating a displacement map from an image sequence. The method 1700 includes an operation 1702 of acquiring a plurality of ultrasound images. In some examples, the three ultrasound images are acquired at operation 1702. In other examples, more than three ultrasound image or fewer than three ultrasound images are acquired.

Next, the method 1700 includes an operation 1704 of creating sub-aperture images for each of the acquired ultrasound images. In some examples, four sub-aperture images are created for each of the acquired ultrasound images. Thus, when three ultrasound images are acquired at operation 1702, a total of 12 sub-apertures are created at operation 1704.

Next, the method 1700 includes an operation 1706 of generating image pyramids for each of the sub-aperture images created from operation 1704. In some examples, the image pyramids have three levels of smoothing and subsampling. In other examples, more than or fewer than three levels of smoothing and subsampling is done. In some examples, a Gaussian image pyramid is constructed by using a Gaussian average for smoothing and subsampling by a factor of two. In these examples, a low-pass filter is applied using the Gaussian image pyramid. In other alternative examples, different types of image pyramids can be generated such as a Laplacian image pyramid in which a band-pass filter is applied.

Next, the method 1700 includes an operation 1708 of calculating tissue displacement from Level 2 images of the image pyramids for each sub-aperture region using image processing techniques on the sub-aperture images. In some examples, the tissue displacement can be estimated by using different displacement estimation techniques such as speckle tracking.

Next, the method 1700 includes an operation 1710 of creating a tissue displacement map for the Level 0 images from the tissue displacements from each sub-aperture image. Mapping tissue displacement values from a Level 2 image to a Level 0 image may include direct mapping of a value of a Level 2 image pixel to a Level 0 pixel neighborhood (4×4 region). As an example, tissue displacement for a corner pixel (0^{th} row, 0^{th} column) of a Level 2 image (d₀₀²) is used to set the tissue displacement values in the Level 0 image 4×4 pixel neighborhood of d₀₀⁰, d₀₁⁰, d₀₂⁰, d₀₃⁰, d₁₀⁰, d₁₁⁰, d₁₂⁰, d₁₃⁰, d₂₀⁰, d₂₁⁰, d₂₂⁰, d₂₃⁰, d₃₀⁰, d₃₁⁰, d₃₂⁰, and d₃₃⁰. Alternative mapping techniques may further include smoothing at Level 0 pixel neighborhood edges. As an example of smoothing, the tissue displacement values within a Level 0 4×4 pixel neighborhood can be linearly interpolated in one direction with neighboring 4×4 pixel neighborhoods. The tissue displacement value d₀₁⁰ is calculated as ¾×d₀₀⁰+¼×d₀₄⁰. The tissue displacement value d₀₂⁰ is calculated as ½×d₀₀⁰+½×d₀₄⁰. The tissue displacement value d₀₃⁰ is calculated as ¼×d₀₀⁰+¾×d₀₄⁰. Alternatively, the tissue displacement values can be bi-linearly interpolated between 4×4 pixel neighborhoods where the tissue displacement values are linearly interpolated in one direction and then linearly interpolated in a second direction.

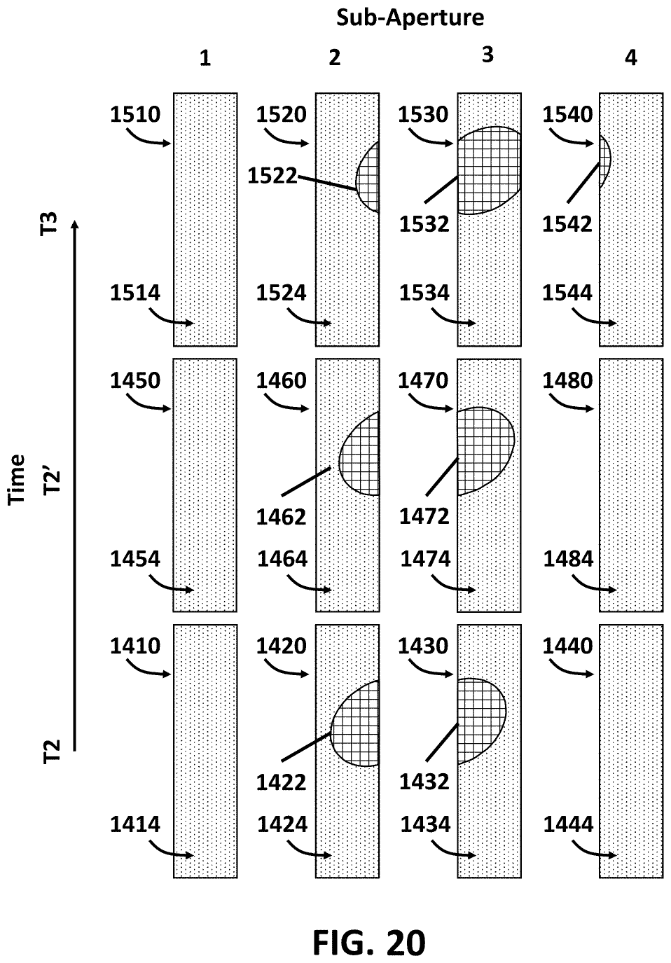

In another example embodiment of the present application, motion estimation techniques can be used to interpolate between sequentially acquired ultrasound images. FIG. 20 illustrates four sub-aperture images 1410, 1420, 1430, and 1440 of the second ultrasound image 1400 at time T2. The four sub-aperture images 1510, 1520, 1530, and 1540 of the third ultrasound image 1500 at time T3 are also shown. Sub-aperture images at a time T2′ where T2<T2′<T3 can be calculated by interpolation of the sub-aperture images at times T2 and T3.

For example, a first sub-aperture image 1450 at time T2′ including a surrounding tissue 1454 is calculated using the first sub-aperture image 1410 at time T2 and the first sub-aperture image 1510 at time T3. A second sub-aperture image 1460 at time T2′ including a target of interest segment 1462 and a surrounding tissue 1464 is calculated using the second sub-aperture image 1420 at time T2 and the second sub-aperture image 1520 at time T3. A third sub-aperture image 1470 at time T2′ including a target of interest segment 1472 and a surrounding tissue 1474 is calculated using the third sub-aperture image 1430 at time T2 and the third sub-aperture image 1530 at time T3. A fourth sub-aperture image 1480 at time T2′ including a surrounding tissue 1484 is calculated using the fourth sub-aperture image 1440 at time T2 and the fourth sub-aperture image 1540 at time T3. The locations of the target of interest segments 1462, 1472 at time T2′ are interpolated between the locations of the target of interest segments 1422, 1432 at time T2 and the locations of the target of interest segments 1522, 1532, 1542 at time T3.

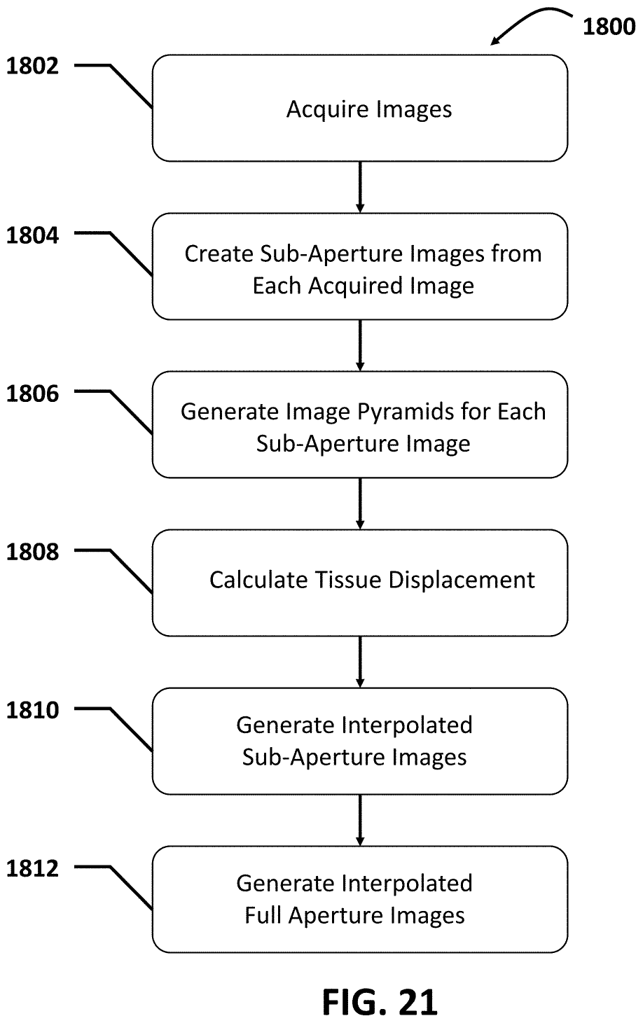

FIG. 21 illustrates a method 1800 for calculating an interpolated ultrasound image from two sequentially acquired ultrasound images. At operation 1802, two ultrasound images are acquired. In other examples, more than two images can be acquired at operation 1802

Next, the method 1800 includes an operation 1804 of generating sub-aperture images from each ultrasound image. In some examples, four sub-aperture images are generated from each acquired ultrasound image. In other examples, more than or fewer than four sub-aperture images are generated from each acquired ultrasound image.

Next, the method 1800 includes an operation 1806 of generating image pyramids for each sub-aperture image. In examples where four sub-aperture images are generated from each ultrasound image, eight image pyramids are generated at operation 1806. In some examples, the image pyramids have three levels of smoothing and subsampling. In other examples, more than or fewer than three levels of smoothing and subsampling is done. In some examples, Gaussian image pyramids are constructed by using a Gaussian average for smoothing and subsampling by a factor of two. In these examples, low-pass filters are applied using the Gaussian image pyramid. In other alternative examples, different types of image pyramids can be generated such as Laplacian image pyramids in which band-pass filters are applied.

The method 1800 further includes an operation 1808 of calculating tissue displacement. In some examples, the tissue displacement is calculated from Level 2 images in each image pyramid. In some examples, tissue displacement is calculated using image processing techniques. Different displacement estimation techniques can be used such as speckle tracking.

Next, the method 1800 includes an operation 1810 of generating interpolated sub-aperture images using the calculated tissue displacements and the Level 0 images. In some examples, the generated interpolated sub-aperture images resemble the first sub-aperture image 1450 at time T2′, the second sub-aperture image 1460 at time T2′, the third sub-aperture image 1470 at time T2′, and the fourth sub-aperture image 1480 at time T2′ shown in FIG. 20.

Next, the method 1800 includes an operation 1812 of generating an interpolated full aperture image by combining the interpolated sub-aperture images. In other examples, different techniques can be used to combine the sub-aperture images into a full aperture image. For example, the Level 2 images in each image pyramid can be combined to create a full aperture image instead of combining the Level 0 images in each image pyramid.

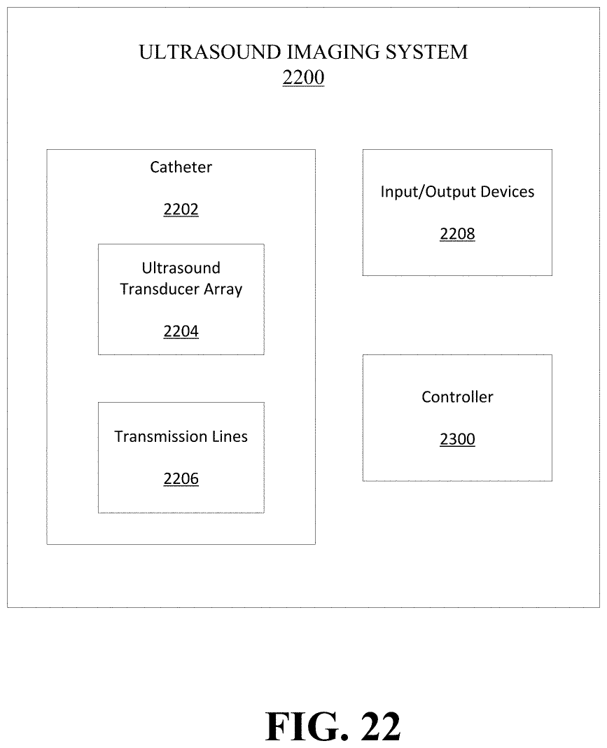

FIG. 22 is a block diagram schematically illustrating an ultrasound imaging system 2200. The ultrasound imaging system 2200 includes a catheter 2202 having one or more ultrasound transducer arrays 2204 and one or more transmission lines 2206. The ultrasound imaging system 2200 can also further include one or more input/output devices 2208 and a controller 2300. In some examples, the one or more input/output devices 2208 and controller 2300 are remotely located from the catheter 2202 such as in an external monitoring console or device.

Each ultrasound transducer array 2204 has a plurality of transducer elements. For example, each ultrasound transducer array 2204 can have 64 transducer elements, 32 transducer elements, or 16 transducer elements. The one or more transmission lines 2206 are programmably connected to the plurality of transducer elements in each ultrasound transducer array 2204. The number of transducer elements in each ultrasound transducer array 2204 is greater than the number of transmission lines 2206, and a programmable connection between the transmission lines and the plurality of transducer elements defines a synthetic aperture size.

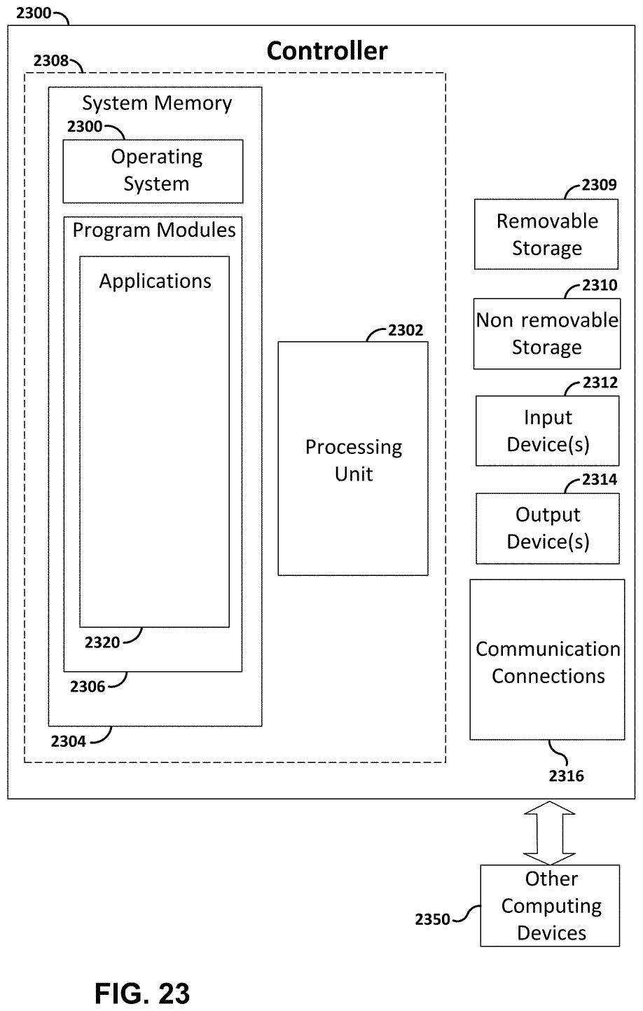

FIG. 23 is a block diagram illustrating physical components (i.e., hardware) of a controller 2300 with which embodiments of the disclosure may be practiced. In a basic configuration, the controller 2300 may include at least one processing unit 2302 and a system memory 2304. The system memory 2304 may include, but is not limited to, volatile storage (e.g., random access memory), non-volatile storage (e.g., read-only memory), flash memory, or any combination of such memories. The system memory 2304 may include an operating system 2305 and one or more program modules 2306 suitable for running software applications 2320. This basic configuration is illustrated in FIG. 23 by those components within a dashed line 2308.

A number of program modules 2306 and data files may be stored in the system memory 2304. While executing on the at least one processing unit 2302, the program modules 2306 may perform various methods and processes including, but not limited to, the methods described with reference to the figures as described herein.

The controller 2300 may have additional features or functionality. For example, the controller 2300 may also include additional data storage devices (removable and/or non-removable) such as, for example, magnetic disks, optical disks, or tape. Such additional storage is illustrated by a removable storage device 2309 and a non-removable storage device 2310.

The controller 2300 may also have one or more input device(s) 2312, such as a keyboard, a mouse, a pen, a sound or voice input device, a touch or swipe input device, etc. Output device(s) 2314 such as a display, speakers, a printer, etc. may also be included. The aforementioned devices are examples and others may be used.

The controller 2300 may also include one or more communication connections 2316 allowing communications with other computing devices 2350. Examples of suitable communication connections 2316 include, but are not limited to, RF transmitter, receiver, and/or transceiver circuitry; universal serial bus (USB), parallel, and/or serial ports.

The term computer readable media as used herein may include non-transitory computer storage media. Computer storage media may include volatile and nonvolatile, removable and non-removable media implemented in any method or technology for storage of information, such as computer readable instructions, data structures, or program modules. The system memory 2304, the removable storage device 2309, and the non-removable storage device 2310 are all computer storage media examples (i.e., memory storage.) Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the controller 2300. Any such computer storage media may be part of the controller 2300. Computer storage media does not include a carrier wave or other propagated or modulated data signal.

The block diagrams depicted in this application are just examples. There may be many variations to these block diagrams without departing from the spirit of the disclosure. For instance, components may be added, deleted or modified. Further, the description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application.

The various embodiments described above are provided by way of illustration only and should not be construed to limit the claims attached hereto. Those skilled in the art will readily recognize various modifications and changes that may be made without following the example embodiments and application illustrated and described herein, and without departing from the true spirit and scope of the following claims.

What is claimed is:

1. An ultrasound imaging system, comprising:
an ultrasound transducer array having a plurality of transducer elements;
a catheter having one or more transmission lines programmably connected to the plurality of transducer elements, the programmable connection between the transmission lines and the plurality of transducer elements defining a synthetic aperture size, and
a controller having at least one processing unit and a system memory storing instructions that, when executed by the at least one processor, causes the ultrasound imaging system to:
acquire images using an initial synthetic aperture size;
detect a relative motion of a target of interest in the acquired images; and
adjust the synthetic aperture size based on the detected relative motion.

2. The system of claim 1, wherein the synthetic aperture size increases when the detected motion is less than a threshold value.

3. The system of claim 1, wherein the synthetic aperture size increases by a factor of two when the detected motion is less than a threshold value.

4. The system of claim 3, wherein the synthetic aperture size increases from 16-elements to 32-elements or from 32-elements to 64-elements based on the detected motion.

5. The system of claim 2, wherein the synthetic aperture size is not adjusted when the detected motion is greater than the threshold value.

6. The system of claim 2, wherein the synthetic aperture size decreases when the detected motion is greater than the threshold value.

7. The system of claim 6, wherein the synthetic aperture size decreases from 64-elements to 32-elements or from 32-elements to 16-elements based on the detected motion.

8. The system of claim 1, wherein the relative motion of the target of interest is detected by generating an image pyramid for each acquired image, calculating pixel-wise and image-wise standard deviations from lower-level images of the image pyramids, and calculating motion weight factors from the image-wise standard deviations.

9. The system of claim 8, wherein the acquired images are filtered using motion weight factors.

10. The system of claim 8, wherein a sequence of three images is acquired, an image pyramid is generated for each acquired image, and each image pyramid has three levels of images in which smoothing and subsampling by a factor of two is repeated two times.

11. A method of acquiring ultrasound images comprising:
acquiring a sequence of images using an initial synthetic aperture size defined by a programmable connection between one or more transmission lines and a plurality of transducer elements;
detecting a relative motion of a target of interest in the acquired images;
maintaining the initial synthetic aperture size when the detected motion is greater than a threshold value; and
increasing the initial synthetic aperture size when the detected motion is less than a threshold value.

12. The method of claim 11, wherein the synthetic aperture size increases by a factor of two when the detected motion is less than the threshold value.

13. The method of claim 11, wherein the synthetic aperture size increases from 16-elements to 32-elements or from 32-elements to 64-elements when the detected motion is less than the threshold value.

14. The method of claim 11, wherein the relative motion is detected by:
generating an image pyramid for each acquired image;
calculating pixel-wise and image-wise standard deviations from lower-level images in each image pyramid; and
calculating motion weight factors from the image-wise standard deviations.

15. The method of claim 11, further comprising filtering the acquired images using motion weight factors calculated from image-wise standard deviations of lower-level images in the image pyramids generated for each acquired image.

16. An ultrasound imaging system for optimizing ultrasound images of a moving target of interest comprising:
an ultrasound transducer array having a plurality of transducer elements;
a catheter having one or more transmission lines operatively connected to the plurality of transducer elements in the ultrasound transducer array; and
a controller having at least one processing unit and a system memory storing instructions that, when executed by the at least one processor, causes the ultrasound imaging system to:
acquire a sequence of images from the ultrasound transducer array;
generate image pyramids for each acquired image;
calculate pixel-wise and image-wise standard deviations from lower-level images of the image pyramids;
calculate motion weight factors from the image-wise standard deviations; and
filter the acquired images using motion weight factors.

17. The system of claim 16, wherein the instructions, when executed by the at least one processor, further cause the ultrasound imaging system to increase a synthetic aperture size defined between the one or more transmission lines and the plurality of transducer elements when a level of detected motion for the target of interest is below a threshold.

18. The system of claim 17, wherein the synthetic aperture size increases by a factor of two.

19. The system of claim 17, wherein the synthetic aperture size increases from 16-elements to 32-elements or from 32-elements to 64-elements.

20. The system of claim 17, wherein the synthetic aperture size decreases when the detected motion is greater than the threshold.
